(19) United States
(12) Patent Application Publication
(10) Pub. No.: US 2008/0286774 A1
(43) Pub. Date: Nov. 20, 2008

(54) Real-time individualized therapy evaluation

(75) Inventors: Kenneth W. Turteltaub, Livermore, CA (US); Paul T. Henderson, Dublin, CA (US); Sang Soo Hah, Dublin, CA (US)

Correspondence Address: Lawrence Livermore National Security, LLC, LAWRENCE LIVERMORE NATIONAL LABORATORY, PO BOX 808, L-703, LIVERMORE, CA, 94551-0808, US

(73) Assignee: The Regents of the University of California

(21) Appl. No.: 11/821,174

(22) Filed: Jun. 21, 2007

Related U.S. Application Data
(60) Provisional application No. 60/930,584, filed on May 16, 2007.

(51) Int. Cl. G01N 21/64; C12Q 1/68; G01N 33/52; G01N 33/96

(52) U.S. Cl. 435/6; 435/40.52; 436/171; 436/94

(57) ABSTRACT

A method of individually optimizing drug therapy to a patient that includes the steps of administering a dose of a radiolabeled drug or fluorescent tags or drugs with inherent fluorescent properties to the patient in connection with chemotherapy, collecting a sample from the patient, analyzing the sample producing an analysis, and using the analysis for developing a model for the patient.

### CROSS-REFERENCE TO RELATED APPLICATION

This application claims the benefit of U.S. Provisional Patent Application No. 60/930,584 filed May 16, 2007 and titled “Real-Time Individualized Therapy Evaluation.” U.S. Provisional Patent Application No. 60/930,584 filed May 16, 2007 and titled “Real-Time Individualized Therapy Evaluation” is incorporated here in this reference.

The United States Government has rights in this invention pursuant to Contract No. W-7405-ENG-48 between the United States Department of Energy and the University of California for the operation of Lawrence Livermore National Laboratory.

### BACKGROUND

1. Field of Endeavor

The present invention relates to therapy evaluation and more particularly to real-time individualized therapy evaluation.

2. State of Technology

U.S. Pat. No. 5,209,919 issued to Kenneth W. Turteltaub et al May 11, 1993 describes and claims a method of quantifying molecules in biological substances, comprising the steps of (a) selecting a biological host comprising biological substances in which radioisotopes are present in concentrations equal to or less than the concentration in the ambient biosphere, (b) preparing a radioisotope-labeled reactive chemical specie, (c) administering said chemical specie to said biological host in doses sufficiently low to avoid significant damage to the host's biological system, (d) allowing a period of time to elapse sufficient for dissemination and reaction of said chemical specie with said host throughout said biological system of said host, (e) isolating a reacted fraction of a sample of biological substance from said host in a manner sufficient to avoid contamination of the fraction from extraneous sources of the radioisotope, (f) converting said fraction of biological substance to a product material which efficiently produces charged ions in an ion source without introduction of significant isotopic fractionation, and (g) measuring the radioisotope concentration in the product material using an accelerator mass spectrometer. U.S. Pat. No. 5,209,919 is incorporated herein by reference.

U.S. Pat. No. 5,366,721 issued to Kenneth W. Turteltaub et al Nov. 22, 1994 describes and claims a method for detection of long-lived radioisotopes in small biochemical samples, comprising: (a) selecting a biological host in which selected radioisotopes are present at a concentration equal to or less than the concentration in the ambient biosphere, (b) preparing a long-lived radioisotope-labeled chemical specie, (c) administering said chemical specie to said biological host in one or more doses that do not cause either significant damage to the host's biological system or a significant increase in radiological exposure to the host over the ambient radiation environment or from natural internal sources of radiation, (d) allowing dissemination and reaction of said chemical specie with said biological system of said host, (e) isolating a reacted fraction of a biological substance from said host in a manner sufficient to avoid contamination of the fraction from extraneous sources of the radioisotope, (f) converting said fraction of biological substance to a solid, inert, non-volatile, thermally conductive inorganic sample which efficiently produces charged ions in an ion source without introduction of significant isotopic fractionation, and (g) measuring the radioisotope concentration in the inorganic sample using an accelerator mass spectrometer. U.S. Pat. No. 5,366,721 is incorporated herein by reference.

U.S. Pat. No. 5,376,355 issued to Kenneth W. Turteltaub et al Dec. 24, 1994 describes and claims a method of assay which comprises: (a) growing a living organism on a diet of food derived from materials having less than natural, present day biospheric levels of a long-lived radioisotope, (b) administering one or more doses of a xenobiotic substance labeled with the radioisotope to said organism, (c) harvesting selected tissues from said organism at selected time points after administration of said doses, (d) extracting DNA from said tissues to remove unbound radioisotopes therefrom, (e) converting said DNA to a material which efficiently produces charged ions in an ion source without introducing significant isotopic fractionation, and (f) measuring the radioisotope concentration of the radioisotope in said material using an accelerator mass spectrometer. U.S. Pat. No. 5,376,355 is incorporated herein by reference.

### SUMMARY

Features and advantages of the present invention will become apparent from the following description. Applicants are providing this description, which includes drawings and examples of specific embodiments, to give a broad representation of the invention. Various changes and modifications within the spirit and scope of the invention will become apparent to those skilled in the art from this description and by practice of the invention. The scope of the invention is not intended to be limited to the particular forms disclosed and the invention covers all modifications, equivalents, and alternatives falling within the spirit and scope of the invention as defined by the claims.

It has been estimated that approximately 25% of drug therapies fail due to individual variation in drug processing, suggesting that individualization of drug treatment could potentially benefit a large number of patients worldwide. Individually typing for optimal drug therapy is not presently done routinely; rather physicians rely on use of common dosing guidelines which do not optimally take into consideration individual differences in body type, size, genetic makeup or drug processing phenotype.

The present invention provides a method for individually optimizing drug therapy to a patient. The method includes the steps of administering a dose of a radiolabeled drug or fluorescent tags or drugs with inherent fluorescent properties to the patient in connection with chemotherapy, collecting a sample from the patient, analyzing the sample producing an analysis, and using the analysis for developing a model for the patient. The present invention provides a methodology to individually optimize drug therapy for disease treatment. The present invention also provides a methodology that can be used to discover novel genes involved in drug absorption, distribution, metabolism, action and elimination.

In one embodiment the present invention involves administration of a miniscule quantum of a radiolabeled, or otherwise labeled, drug or drug cocktail, followed by a few repeated blood samplings and analysis of the sample by accelerator mass spectrometry (AMS) or technology appropriate for the physical-chemical properties of the labeled drug. Blood samples are collected by veinopuncture, biopsy or pin prick and may or may not, depending on the diagnostic test, be separated into individual drug metabolites by an appropriate separatory technique prior to analysis. The quantity of drug or drug metabolite will be indicated by the AMS or other analysis. This analysis will indicate the key drug dose and dosing interval to be used in treating a patient. One step in the diagnosis is the use of very low levels of labeled drug followed by analysis with an accelerator mass spectrometer or other technique and conversion of the data to a “concentration×time” curve which a clinician can use to prescribe the correct drug type, dose and dosing interval needed to attain the optimal therapeutic dose at the target site. The method constitutes a rapid cost effective approach to individually tailor drug therapy, screen for therapy effectiveness and minimizes side effects resulting from drug therapy.

The present invention has many uses. One use of the invention is to tailor drug dose and dosing interval for cancer chemotherapy. Four broad uses for the present invention are the following: (1) determining whether a drug will be useful in a person, (2) defining the optimal dose to give the person, (3) monitoring during therapy for development of drug resistance of changes in drug processing that would affect efficacy, and (4) discovery of new candidate drug target genes.

One use is a point-of-care diagnostic to assess prognosis or predict the likelihood of a positive therapeutic outcome. Differences in a person's ability to process a drug may affect ability to interact with its therapeutic target. Examples of this are specific mutations in tumors that drugs are targeted against. Testing for the ability of a DNA damaging drug to be repaired by the cell will effect its cytotoxicity so a person with a high degree of DNA repair for that drug-DNA adduct will result in lower tumor cell killing and less efficacy.

Another use is as a point-of-care diagnostic that will phenotype individual patients for their ability to process a drug. This information is important to selecting the correct drug, drug dosage and dosing interval to maintain the therapeutic dose in the target or diseased tissue. It is also important for minimizing any deleterious effects of the drug to help maintain the maximal quality of life while a patient is on the drug therapy. For this application there is a particular need for speed, small sample size and use of low amounts of labeled drug during the diagnostic period.

Another use is as a point-of-care diagnostic for drug resistance after a therapy has commenced. This information is critical in cancer chemotherapy to assure that a patient is continuously on the most appropriate drug to kill the tumor and is removed from any drug for which the tumor has become resistant as early as possible. This is presently based on x-ray or other tomography-based methods which monitors for changes in tumor size. A decreased rate of tumor shrinkage or an enlargement of tumor size while on drug therapy indicates drug resistance. The method proposed here potentially can lead to earlier detection of drug resistance based on the microscopic changes in drug disposition rather than waiting for observable macroscopic changes in tumor mass.

Another use is as a discovery test to find and characterize genes involved in drug metabolism and consequently targets for new diagnostics of therapeutic prognosis and new targets for therapeutics.

The invention is susceptible to modifications and alternative forms. Specific embodiments are shown by way of example. It is to be understood that the invention is not limited to the particular forms disclosed. The invention covers all modifications, equivalents, and alternatives falling within the spirit and scope of the invention as defined by the claims.

### BRIEF DESCRIPTION OF THE DRAWINGS

The accompanying drawings, which are incorporated into and constitute a part of the specification, illustrate specific embodiments of the invention and, together with the general description of the invention given above, and the detailed description of the specific embodiments, serve to explain the principles of the invention.

FIG. 1 is a schematic of an accelerator mass spectrometry (AMS) system.

FIG. 2 illustrates an embodiment of a method for individually optimizing drug therapy to a patient using accelerator mass spectrometry.

FIG. 3 illustrates an embodiment of a method for individually optimizing drug therapy to a patient using sensitive fluorimeters.

### DETAILED DESCRIPTION OF THE INVENTION

Referring to the drawings, to the following detailed description, and to incorporated materials, detailed information about the invention is provided including the description of specific embodiments. The detailed description serves to explain the principles of the invention. The invention is susceptible to modifications and alternative forms. The invention is not limited to the particular forms disclosed. The invention covers all modifications, equivalents, and alternatives falling within the spirit and scope of the invention as defined by the claims.

The action or effect of a drug is mediated mechanistically through the specific interaction of the drug with a molecular target in the body. It is commonly believed that the intensity of the action is a function of the amount of drug reaching the target molecule and the amount of drug reaching the target molecule is related to the dosage, absorption, distribution, biotransformation and excretion. Thus, developing methods that allow the rapid quantification of the “time×drug” concentration response at the site of action in a person will allow for adjustment of the dosage to ensure that the correct amount of the biologically-active drug is present to do its work.

### Optimizing Drug Therapy Using Accelerator Mass Spectrometry

Applicants believe that such time-resolved drug quantification can be carried out using extremely small amounts of isotope-labeled drugs that can be given to patients and quantified through use of the unique technology of accelerator mass spectrometry (AMS). This is also possible using other tags, either added to the drug molecule, or an inherent physicochemical property of the molecule itself. Examples include the use of fluorescent tags or drugs with inherent fluorescent properties followed by analysis with sensitive fluorimeters. Isotopes can be analyzed by other methods as well, depending on the isotope, such as scintillography if the situation warrants. Thus the method could utilize any type of detectable tag and analysis method appropriate for the properties of the molecule.

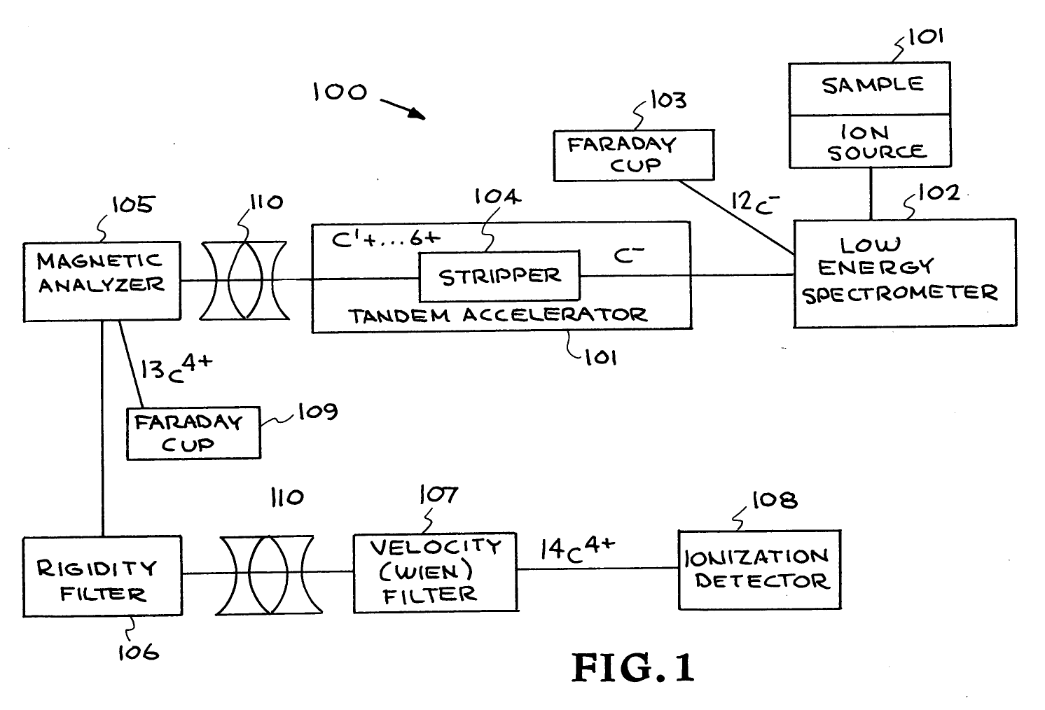

Referring now to the drawings and in particular to FIG. 1, a schematic of an accelerator mass spectrometry (AMS) system is shown. The AMS system is designated generally by the reference numeral 100. Systems for accelerator mass spectrometer (AMS) are described in U.S. Pat. No. 5,209,919; U.S. Pat. No. 5,366,721; and U.S. Pat. No. 5,376,355. U.S. Pat. No. 5,209,919; U.S. Pat. No. 5,366,721; and U.S. Pat. No. 5,376,355 are incorporated herein by reference.

AMS is a technique for measuring isotope ratios with high selectivity, sensitivity, and precision. In general, AMS separates a rare radioisotope from stable isotopes and molecular ions of the same mass using a variety of nuclear physics techniques. In the case of carbon, ¹⁴C ions are separated and counted as particles relative to ¹³C or ¹²C that are measured as an electrical current. The key steps of AMS allowing quantitative and specific measurement of isotopes are the production of negative ions from the sample to be analyzed, a molecular disassociation step to convert the negatively charged molecular ions to positively charged nuclei and the use of high energies (MeV) which allow for the identification of ions with high selectivity.

Radiocarbon AMS analysis requires samples be converted to a form that retains the isotopic ratio from the original sample and that provides chemical and physical equivalence for all carbon atoms. Consequently, ¹⁴C AMS currently uses mg sized graphite aliquots derived from the CO₂ of oxidized samples as the interface between biological samples and AMS measurement of ¹⁴C. These samples are bombarded by a Cs⁺ beam forming negative elemental or molecular ions. The production of negative ions removes the primary isobaric interference for radiocarbon, ¹⁴N, because nitrogen does not form a stable negative ion. Ions are subsequently selected at single atomic mass units through a low energy magnetic analyzer by switching the electrostatic potential on the magnets vacuum chamber. ¹²C is separated from mass 13 and 14 ions and can be quantified in a Faraday cup. This low energy mass spectrometer cannot resolve the small differences among the rare isotope and the nuclear and molecular isobars. Hence negative ions and molecules are accelerated to MeV energies in the first stage of a tandem accelerator 101. At the end of this first acceleration stage these ions pass through a stripper 104. The stripper, which consists of a thin carbon foil or gas, strips electrons from ions and destroys molecular isobars, such as ¹³CH. These positive ions are further accelerated to energies of up to several tens of MeV in the second stage of the tandem accelerator. Acceleration of the ions to high energies makes possible the unique identification of ions based on energy loss and total energy.

Positive nuclei from the accelerator are focused by a quadruple lens 110, and then separated on the basis of momentum by a magnetic analyzer 105. ¹³C is measured after this first high energy magnetic analyzer in a Faraday cup 105. Nuclei of the correct rigidity (momentum/charge) are then passed through a second magnetic analyzer to reject scattered ions of incorrect mass/charge, refocused by a second quadrupole lens 110, passed through a Wein filter 107 to select for velocity, and finally counted in a multi-anode gas-ionization detector 108. Importantly AMS counts ¹⁴C as individual nuclei and is independent of decay. Around 1% of the ¹⁴C in a sample is counted which is 1000-times more efficient than decay counting. Sensitivities approaching ¹⁴C/C of ˜2×10⁻¹⁵ or 10 attomoles of ¹⁴C can be achieved in mg sized samples.

AMS is a technique that measures isotope ratios at extremely low levels. Application of AMS allows use of drugs at concentrations so low as to be considered non-radioactive and non-toxic. This can be accomplished using tissue samples obtained from needle biopsy or in μl-sized blood samples. This method can quantify attomoles (10⁻¹⁸ moles) of a drug in real samples and with radiological doses of a few hundred nanocuries per person.

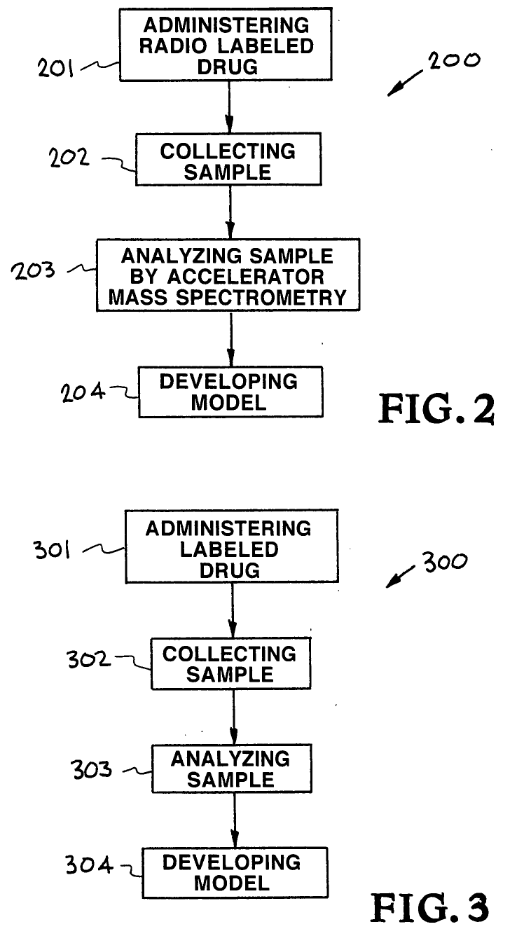

Referring now to FIG. 2 one embodiment of a method for individually optimizing drug therapy to a patient is illustrated by a flow diagram. The method for individually optimizing drug therapy to a patient is designated generally by the reference numeral 200. The method includes the following steps:

Step 1—Administering a dose of a radiolabeled drug to the patient in connection with chemotherapy (Designated by the reference numeral 201).

Step 2—Collecting a sample from the patient (Designated by the reference numeral 202).

Step 3—Analyzing the sample by accelerator mass spectrometry producing an analysis (Designated by the reference numeral 203).

Step 4—Using the analysis for developing a model for the patient (Designated by the reference numeral 204).

An advantage of this approach relative to other techniques such as functional NMR, for example, is that it will broaden the range of compounds that the methodology can be utilized for. This approach will not require a compound that is NMR or optically active but will be applicable to any compound with which a stable isotopic label can be added. Relative to PET, it does not require the use of freshly produced substrates as the radioisotopic tag has much longer lifetimes. It also offers the promise of providing increased sensitivity for quantification of lower amounts of drug (AMS can detect attomolar levels of a compound and is quantitative).

This methodology is coupled to pharmacokinetic modeling so that a test dose can be applied to an individual and used to establish the optimal dose and dosing interval to attain the required drug concentration at the target site and to assess when drug resistance has developed based on changes in the kinetics of drug metabolism. Additionally, Applicants believe utilizing this methodology to study individuals and stratify them based on pharmacokinetic response into groups that can be studied using genomic techniques to identify the important genes involved. It will also allow for characterization of the mutations in those genes that affect the drugs pharmacokinetic profile. Elucidation of these genes can then be used to establish pathways and to predict cellular response to drugs.

The present invention provides a method using accelerator mass spectrometry and pharmacokinetic modeling to determine an individual's phenotype for drug absorption, distribution metabolism and elimination phenotype and to use that phenotype to individually set the optimal dose to administer and how often to administer it. The method offers an applicable improvement over current standard medical practice to rely on general guidelines for setting drug dosage which is primarily based on body weight or mass and ignores individual differences in metabolism, background and genetics.

Briefly, the methodology relies on administering a small dosage of a radiolabeled drug prior to initiation of chemotherapy or during the actual therapy itself. At selected time intervals a small blood or other sample is collected from the patient and analyzed by AMS. Analysis by AMS can be preceded by application of a separatory method to speciate the individual drug metabolites if it is needed to differentiate pro and active drug. Results from the analysis are applied to develop a pharmacokinetic model for the patient and used to select optimal drug dose, type and dosing interval. Additional samples are collected from the patient after administering small doses of radiolabeled drug during the therapy to continuously monitor the status of the therapy and detect any changes in the metabolic and kinetic profile to signify development of resistance to the drug.

In a similar fashion, rather than analyze for soluble drug metabolites, tissue samples taken from the patient after administration of the radiolabeled drug can be used to quantify the amount of DNA-drug adduct formed for drugs designed to bind to DNA. The ability of the patient to repair the therapeutic DNA adducts is a prognostic indicator of the therapy. Measurement of metabolites, pharmacokinetics DNA adducts and the methods used for analysis of biological samples by AMS have all been developed by Applicants.

### Optimizing Drug Therapy Using Sensitive Fluorimeters

Referring now to FIG. 3 an embodiment of a method for individually optimizing drug therapy to a patient using sensitive fluorimeters is illustrated by a flow diagram. The method for individually optimizing drug therapy to a patient using sensitive fluorimeters is designated generally by the reference numeral 300. The method includes the following steps:

Step 1—Administering a miniscule quantum of a radiolabeled, or otherwise labeled, drug or drug cocktail, to the patient in connection with chemotherapy (Designated by the reference numeral 301).

Step 2—Collecting a sample from the patient (Designated by the reference numeral 302).

Step 3—Analyzing the sample by a technology appropriate for the physical-chemical properties of the labeled drug producing an analysis (Designated by the reference numeral 303).

Step 4—Using the analysis for developing a model for the patient (Designated by the reference numeral 304).

Briefly, the methodology relies administering a small dosage of a labeled drug prior to initiation of chemotherapy or during the actual therapy itself. At selected time intervals a small blood or other sample is collected from the patient and analyzed. Results from the analysis are applied to develop a pharmacokinetic model for the patient and used to select optimal drug dose, type and dosing interval. Additional samples are collected from the patient after administering small doses of labeled drug during the therapy to continuously monitor the status of the therapy and detect any changes in the metabolic and kinetic profile to signify development of resistance to the drug.

In a similar fashion, rather than analyze for soluble drug metabolites, tissue samples taken from the patient after administration of the labeled drug can be used to quantify the amount of DNA-drug adduct formed for drugs designed to bind to DNA. The ability of the patient to repair the therapeutic DNA adducts is a prognostic indicator of the therapy.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

The invention claimed is:

1. A method of individually optimizing drug therapy to a patient, comprising the steps of:
administering a dose of a radiolabeled drug or fluorescent tags or drugs with inherent fluorescent properties to the patient in connection with chemotherapy,
collecting a sample from the patient,
analyzing said sample producing an analysis, and
using said analysis for developing a model for the patient.

2. The method of individually optimizing drug therapy to a patient of claim 1 wherein said step of analyzing said sample producing an analysis comprises analyzing said sample by accelerator mass spectrometry producing an accelerator mass spectrometry analysis.

3. The method of individually optimizing drug therapy to a patient of claim 1 wherein said step of analyzing said sample producing an analysis comprises analyzing said sample by analyzing said sample with sensitive fluorimeters producing a fluorimeters analysis.

4. The method of individually optimizing drug therapy to a patient of claim 1 wherein said step of analyzing said sample producing an analysis comprises analyzing said sample by accelerator mass spectrometry producing an accelerator mass spectrometry analysis, and wherein said step of using said analysis for developing a model for the patient comprises using said accelerator mass spectrometry analysis for developing a model for the patient.

5. The method of individually optimizing drug therapy to a patient of claim 1 wherein said step of developing a model for the patient comprises developing a pharmacokinetic model for the patient.

6. The method of individually optimizing drug therapy to a patient of claim 5 including using said pharmacokinetic model to select an optimal drug dose, type and dosing interval for the patient.

7. The method of individually optimizing drug therapy to a patient of claim 5 including collecting an additional sample from the patient to continuously monitor the status of the therapy and detect any changes in the metabolic and kinetic profile to signify development of resistance to the drug.

8. The method of individually optimizing drug therapy to a patient of claim 1 wherein said step of developing a model for the patient comprises developing a metabolites model for the patient.

9. The method of individually optimizing drug therapy to a patient of claim 1 wherein said step of developing a model for the patient comprises developing a DNA adducts model for the patient.

10. The method of individually optimizing drug therapy to a patient of claim 9 wherein said step of developing a DNA adducts model for the patient provides information regarding DNA-drug adduct formed for drugs designed to bind to DNA.

11. The method of individually optimizing drug therapy to a patient of claim 1 wherein said step of collecting a sample from the patient comprises collecting a blood sample.

12. The method of individually optimizing drug therapy to a patient of claim 1 wherein said step of collecting a sample from the patient comprises collecting a tissue sample.

13. A method of individually optimizing drug therapy to a patient, comprising the steps of:
administering a dose of a radiolabeled drug to the patient in connection with chemotherapy,
collecting a sample from the patient,
analyzing said sample by accelerator mass spectrometry producing an analysis, and
using said analysis for developing a model for the patient.

14. The method of individually optimizing drug therapy to a patient of claim 13 wherein said step of developing a model for the patient comprises developing a pharmacokinetic model for the patient.

15. The method of individually optimizing drug therapy to a patient of claim 4 including using said pharmacokinetic model to select an optimal drug dose, type and dosing interval for the patient.

16. The method of individually optimizing drug therapy to a patient of claim 4 including collecting an additional sample from the patient to continuously monitor the status of the therapy and detect any changes in the metabolic and kinetic profile to signify development of resistance to the drug.

17. The method of individually optimizing drug therapy to a patient of claim 13 wherein said step of developing a model for the patient comprises developing a metabolites model for the patient.

18. The method of individually optimizing drug therapy to a patient of claim 13 wherein said step of developing a model for the patient comprises developing a DNA adducts model for the patient.

19. The method of individually optimizing drug therapy to a patient of claim 8 wherein said step of developing a DNA adducts model for the patient provides information regarding DNA-drug adduct formed for drugs designed to bind to DNA.

20. The method of individually optimizing drug therapy to a patient of claim 13 wherein said step of collecting a sample from the patient comprises collecting a blood sample.

21. The method of individually optimizing drug therapy to a patient of claim 13 wherein said step of collecting a sample from the patient comprises collecting a tissue sample.
